(19) United States
(12) Patent Application Publication
(10) Pub. No.: US 2015/0257661 A1
(43) Pub. Date: Sep. 17, 2015

(54) SYSTEM AND METHOD FOR DETERMINING ARTERIAL PULSE WAVE TRANSIT TIME

(71) Applicant: Xerox Corporation, Norwalk, CT (US)

(72) Inventors: Lalit Keshav MESTHA, Fairport, NY (US); Survi KYAL, Rochester, NY (US)

(73) Assignee: XEROX CORPORATION, Norwalk, CT (US)

(21) Appl. No.: 14/204,397

(22) Filed: Mar. 11, 2014

(51) Int. Cl. A61B 5/021; A61B 5/026; A61B 5/00; A61B 5/024

(52) U.S. Cl. CPC A61B 5/02125; A61B 5/02427; A61B 5/0261; A61B 5/0015

(57) ABSTRACT

What is disclosed is a system and method for determining the time it takes for an arterial pulse pressure wave to transit between a proximal and a distal point of a patient's body. In one embodiment, a signal is received from each of a first and second device worn circumferentially around a proximal and a distal region, respectively. The devices are worn on an area of exposed skin. Each of the devices comprises at least one emitter/detector pair fixed to an inner side of each device and has at least one detector paired to at least one illuminator. Each detector has at least one sensor that is sensitive to a wavelength band of light emitted by its illuminator. Each device generates signals that are proportional to an intensity of light emitted by an illuminator. The signals are analyzed to determine arterial pulse wave transit time between the proximal and distal regions.

### TECHNICAL FIELD

The present invention is directed to a system and method for determining the time it takes for an arterial pulse pressure wave to transit from a proximal to a distal point of a subject's body such that various arterial functions can be assessed.

### BACKGROUND

The ability to capture physiological signals is highly desirable in the healthcare industry. One physiological signal of importance is the pulse transit time (PTT) for many reasons, one of which is that the PTT correlates well with blood pressure and thus can provide the healthcare professional with information relating to blood velocity in the vascular network, blood vessel dilation over time, and vessel blockage between two points or regions. Moreover, localized PTT can be used as an indirect marker for assessing pathologic conditions such as peripheral vascular disease. Typically, in order to obtain such measurements, electrodes need to be attached to the patient's skin. This can be an issue in a neonatal intensive care unit (NICU) managing care for premature infants with highly sensitive skin.

Accordingly, what is needed in this art are increasingly sophisticated systems and methods for determining arterial pulse transit time in an unobtrusive, non-contact, remote-sensing environment.

### BRIEF SUMMARY

What is disclosed is a system and method for determining the time it takes for an arterial pulse pressure wave to transit between a proximal and a distal point of a patient's body. In one embodiment, a signal is received simultaneously from each of a first and second device worn circumferentially around a proximal and a distal region, respectively, of the body. The devices are worn on an area of exposed skin of a subject being monitored for pulse wave transit time. The signals are analyzed, in a manner more fully disclosed herein, to determine an arterial pulse wave transit time for the subject between the proximal and distal regions. Each of the devices comprises at least one emitter/detector pair fixed to an inner side of each device and has at least one detector paired to at least one illuminator. Each detector has at least one sensor that is sensitive to a wavelength band of light emitted by its illuminator(s). Each emitter/detector pair is separated from its respective paired illuminator(s) by a distance D. Embodiments are disclosed for both a transmissive and reflective sensing emitter/detector pairs. Each device generates signals which are proportional to an intensity of light emitted by its illuminators. The signals are then analyzed to determine an arterial pulse wave transit time for the subject between the proximal and distal regions.

Features and advantages of the above-described system and method will become apparent from the following detailed description and accompanying drawings.

### BRIEF DESCRIPTION OF THE DRAWINGS

The foregoing and other features and advantages of the subject matter disclosed herein will be made apparent from the following detailed description taken in conjunction with the accompanying drawings, in which:

FIG. 1 shows a subject wearing a plurality of reflective or transmissive devices comprising emitter/detector pairs, over differing areas of exposed skin;

FIG. 2 shows one embodiment of a transmissive device comprising a plurality of emitter/detector pairs;

FIG. 3 shows one embodiment of a reflective device comprising a plurality of emitter/detector pairs;

FIG. 4 shows the angular relationships of a emitter/detector pair of the reflective device of FIG. 3;

FIG. 5 shows one embodiment of a control panel fixed to an outer side of both of the devices worn on a proximal and distal area of exposed skin by the subject of FIG. 1;

FIG. 6 illustrates Beer's Law in a tissue layer containing venous and arterial structures;

FIG. 7 is a flow diagram which illustrates one example embodiment of the present method for determining arterial pulse wave transit time;

FIG. 8 is a continuation of the flow diagram of FIG. 7 with flow processing continuing with respect to node A; and

FIG. 9 illustrates a block diagram of one example signal processing system for performing various aspects of the teachings hereof as discussed with respect to the flow diagrams of FIGS. 7 and 8.

### DETAILED DESCRIPTION

What is disclosed is a system and method for determining arterial pulse transit time.

### NON-LIMITING DEFINITIONS

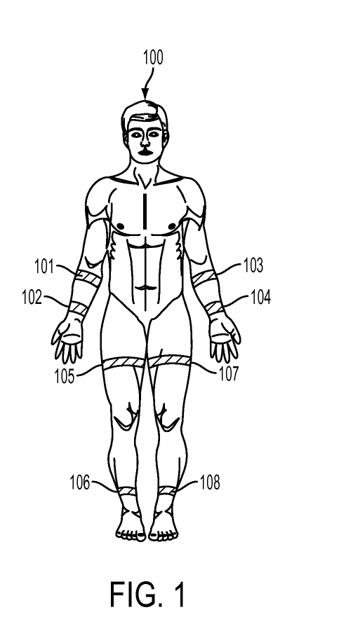

A “subject” refers to a person or patient for which arterial pulse wave transit time is to be determined. Although the terms “person” or “patient” may be used throughout this disclosure, it should be appreciated that the subject may not be human. As such, use of the terms “human”, “person” or “patient” is not to be viewed as limiting the scope of the appended claims strictly to human beings. FIG. 1 shows an example subject 100 wearing a plurality reflective or transmissive devices comprising emitter/detector pairs, over differing areas of exposed skin.

An “arterial pulse wave” is a pressure wave in the vascular system which is generated when the heart muscle pushes a volume of blood into the subject's vascular network. As the left ventricle of the heart contracts, blood exits the ascending aorta in the form of waves and flows into systemic arteries. The push of this blood volume generates a perturbation (a pressure wave) that travels through the arteries. In humans, an arterial pulse pressure wave has two primary components, i.e., a forward wave generated when the left ventricle of the heart muscle contracts, and a reflected wave returning back from the vascular system. Pressure in the aorta is the sum of the forward wave and the reflected wave.

“Pulse wave transit time” (PWTT) (or simply PTT) is the time it takes an arterial pulse pressure wave to transit from a proximal (upstream) point to a distal (downstream) point of the subject's body. Since PTT is pressure wave with a velocity, it is therefore a function of blood pressure, vessel diameter, and blood density. PTT signals can be calibrated to extract beat-to-beat blood pressure and blood velocity in the vascular network. PTT can be used to facilitate a variety of diagnosis including blood vessel dilation over time and vessel blockage between two points or regions. Localized PTT can be used as an indirect marker for assessing various pathologic conditions such as, for instance, peripheral vascular disease. PTT is measured between a proximal and a distal point or region.

“Proximal” (from the Latin proximus: meaning nearest to) refers to a point that is nearer to the source of the arterial pulse which, in humans, is in the ascending aorta. Note that the systemic arterial system originates from the aorta. For arterial pulse measurement purposes, a proximal point in an artery is a point which is closer to the heart than the distal point, i.e., upstream from the distal point.

“Distal” (from the Latin distare: meaning away from) refers to a point that is farther from a center of the body. For arterial pulse measurement purposes, the distal point in the artery is a point which is farther from the heart, for example, downstream from the proximal point. By drawing an imaginary line between the proximal and distal points, a proximo-distal axis is created. A “proximal” device comprising at least one emitter/detector pair and a “distal” device comprising at least one emitter/detector pair are used to determine PTT for the subject.

An “emitter/detector pair” refers to at least one illuminator and at least one detector. The illuminator emits light at a desired wavelength band. In one embodiment, the wavelength band is centered around 660 nm or 940 nm. The photodetector (or simply “detector”) has light sensing elements (sensors) which are sensitive to a wavelength band of the illuminator(s). Each detector outputs a signal which is proportional to an intensity of received light emitted by its illuminator(s). Emitters and detectors are fixed to an inner side of a band worn circumferentially around an area of exposed skin. FIG. 1 shows various areas of exposed skin of the subject 100 where the present devices can be worn. For example, emitter/detector pair 101 is proximal to emitter/detector 102. Emitter/detector pair 103 is proximal to emitter/detector pair 104. Emitter/detector pair 105 is proximal to emitter/detector pair 106. Emitter/detector pair 107 is proximal to emitter/detector pair 107. It should be appreciated that the embodiment of FIG. 1 is illustrative for explanatory purposes and that the emitter/detector pairs can be worn around other proximal and distal locations of the subject 100 where it is desired to determine a pulse wave transit time therebetween. An emitter/detector pair can be either a transmissive or reflective device.

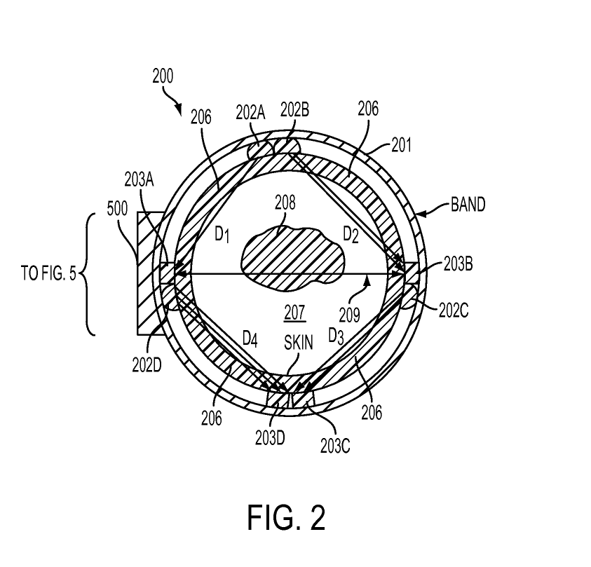

A “transmissive device” measures an intensity of light passing through a chord of living tissue. FIG. 2 shows an embodiment of a transmissive device 400 comprising a band 201 with a plurality of emitter/detector pairs fixed to an inner side thereof. In the embodiment of FIG. 2, emitters 202A, 202B, 202C and 202D are paired, respectively, to detectors 203A, 203B, 203C and 203D. Emitter 202A is shown as a single illuminator which emits source light at a desired wavelength band. Emitters 202B and 202C are shown each comprising two illuminators, which may emit light at the same or different wavelength bands. Emitter 202D is shown comprising three illuminators which may emit light at the same or different wavelength bands. It should be appreciated that band 201 may comprise any configuration of emitter/detector pairs and that the embodiment shown is illustrative. Band 201 is worn circumferentially around an area of exposed skin 206 covering subcutaneous tissues 207 which surround deeper tissues such as muscles, organs, bones, and the like, (collectively at 208). Distances D₁, D₂, D₃ and D₄ define a chord of living tissue through which light emitted by illuminators 202A-D passes. The light is detected by respective photodetectors 203A-D. Although not illustrated to scale, distances D₁, D₂, D₃, D₄ are less than 75% of a diametrical distance (at 209) of the area around which the band is worn. Distances between respective emitter/detector pairs do not have to be equal.

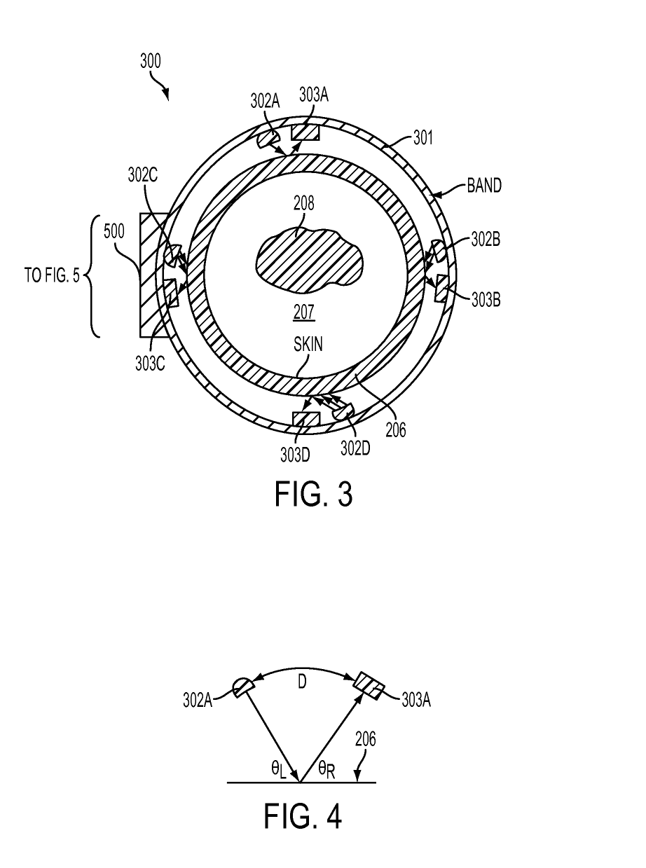

A “reflective device” measures an intensity of light reflecting off the skin surface. FIG. 3 shows one embodiment of a transmissive device 300 comprising a band 301 with a plurality of emitter/detector pairs fixed to an inner side thereof. Emitters 302A-D are paired, respectively, to detectors 303A-D. Emitter 302A comprises a single illuminator which emits source light at a desired wavelength band. Emitters 302B-C each comprise two illuminators which may emit light at the same or different wavelength bands. Emitter 302D is shown comprising three illuminators which may all emit source light at a same or different wavelength bands. It should be appreciated that band 301 may comprise any configuration of emitter/detector pairs and that the embodiment shown is illustrative. Band 301 is worn circumferentially around an area of exposed skin 206 covering a plurality of subcutaneous tissues 207 which surround deeper tissues such as muscles, organs, bones, and the like, (collectively at 208). In FIG. 4, the source light emitted by illuminator 302A impacts the skin surface 206 at angle θ_{L} and reflects off the skin surface at angle θ_{R}, where 0°<(θ_{L}, θ_{R})<90°. A reflection of the light is measured by sensors of detector 303A. Distance D is the distance between illuminator 302A and detector 303A. Distances between respective emitter/detector pairs do not have to be equal.

“Receiving a signal” from a first and second device worn on a proximal and a distal region is intended to be widely construed and includes: retrieving, capturing, acquiring, or otherwise obtaining the signals from the detectors for processing. The signals can be retrieved from a memory or media incorporated into the device. The signals may be communicated to a remote device over a network and received therefrom for processing. The signals may be retrieved from a media such as a CDROM or DVD. The signals may be downloaded from a web-based system or application which makes such measurement signals available for processing. The signals can also be retrieved using an application such as those available for handheld cellular devices and other handheld devices such as an iPad, Smartphone, and Tablet.

“Analyzing the signals” means to process the received signals to determine an arterial pulse wave transit time for the subject. In one embodiment, analyzing the signals involves processing the signals from each of the “proximal” and “distal” devices to generate a time-series signal for each device. For each of the time-series signals, phase angles are computed by unwrapping after every 360 degrees with respect to frequency to obtain respective phase v/s frequency curves. Slopes are extracted from the phase v/s frequency curves within a cardiac frequency range of 0.75 to 4.0 Hz. A difference between the slopes is computed. This difference is the arterial pulse transit time (PTT) for the subject. In another embodiment, the signals are analyzed to determine a phase difference and the phase difference is processed with the subject's heart rate to determine the PTT.

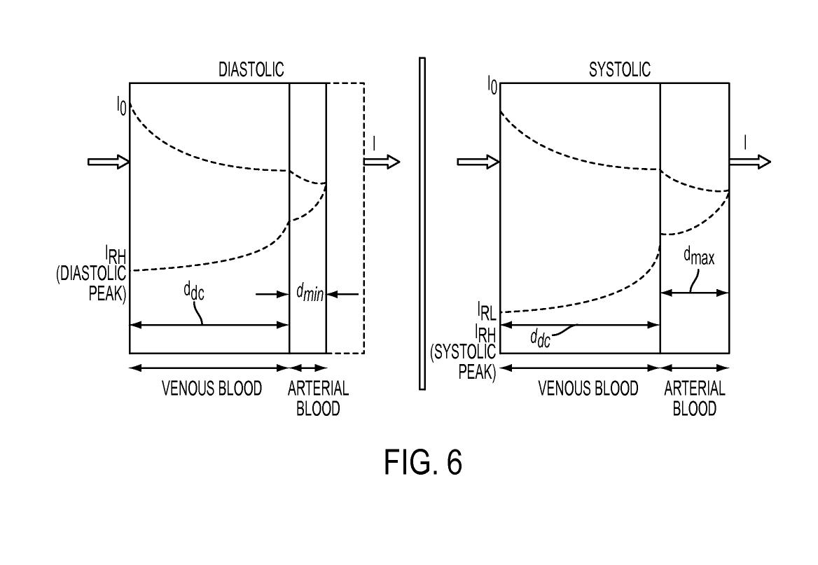

According to Beer's Law (also known as Beer-Lambert-Bouguer's law), as light travels through a media, the intensity I decreases exponentially with distance. FIG. 6 illustrates Beer's Law in a tissue layer containing venous and arterial structures.

For transmissive sensing, if the sensor measures a dc level, V_{dc}, during diastole, and the ac level V_{ac}, during systole (i.e., modulation above the dc level), then the transmittance T can be written as the ratio as follows:

$$T = \frac{{V}_{dc} + {V}_{ac}}{{V}_{dc}} \qquad ( 1 )$$

Signals are pulsating due to volumetric change in blood volume during systolic and diastolic peaks. The dc portion of this signal is due to non-changing entities such as tissue, venous blood, bone, etc.

For reflective sensing, if the sensor measures a dc level, V_{dc}, during diastole, and the ac level V_{ac}, during systole then the reflectance R can be written as:

$$R = \frac{{V}_{dc} + {V}_{ac}}{{V}_{dc}} \qquad ( 2 )$$

### Example Control Panel

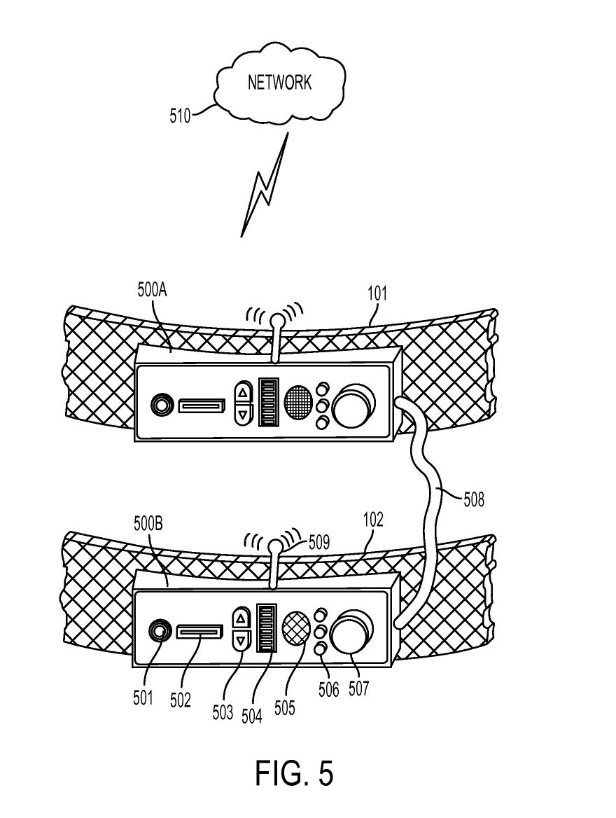

Reference is now being made to FIG. 5 which shows one embodiment of a control panel fixed to an outer side of both of the devices worn on a proximal and distal area of exposed skin by the subject of FIG. 1. In this embodiment, both devices (101 and 102) have a control panel 500A and 500B, respectively, which are similarly configured. The control panel allows the user to effectuate various functionality.

Each of the control panels has an adaptor 501 for receiving a connector to a power supply to charge one or more batteries (not shown) housed therein. A separate power supply comprising a battery pack may be kept in a pocket and a cord connected to the control pattern via adaptor 501. The power supply may be a transformer plugged into a wall socket with a cord which provides continuous power to the device.

Slot 502 enables the insertion of a memory chip or MicroSD card. Such a removable memory card records signals obtained from the detectors, and may contain device specific parameters which are used to set power levels, adjust intensity values, data, formulas, threshold values, patient information, and the like. Once inserted into slot 502, a processor or ASIC internal to the control panel reads the data as needed.

Directional buttons 503 enable a variety of functionality including increasing/decreasing a volume played through speaker 504. Buttons 503 may be configured to increase an intensity of the light emitted by the illuminators. Buttons 503 may be utilized to change a wavelength band of the light emitted by the illuminators, or to adjust a sensitivity of the sensors of the detectors. Buttons 503 may be used to tune the present apparatus to standards set by the FDA or other regulatory agencies.

USB port 504 enables the connection of a USB device to the present device to further enable any of a variety of functions. For example, a USB device may be connected to the present device and used to upload or program a microprocessor or configure the present device to a particular patient. The USB device may be used to set threshold levels or is a memory or storage device which contains one or more threshold levels for PTT detection and monitoring.

Speaker 505 enables an audible feedback for the visually impaired. Such as an audible alert may be initiated in response to the determined PTT being outside a pre-defined limit of acceptability. The audible alert may be varied in volume, frequency, and intensity, as desired, using directional buttons 503.

LEDs 506 enable a variety of visual feedback which may be advantageous for the hearing impaired. For instance, a visual feedback may take the form of a green LED being activated when the device is turned ON, a red LED being activated in response to an alert condition, and a blue LED being activated when the device is operating normally or when an alert condition is not present. The LEDs may be activated in response to a communication occurring between the present device and a remote device via a wireless communication protocol. The LEDs may be activated in combination by turning some lights ON according to a pattern. More LEDs may be utilized.

Button 507 turns the device ON/OFF or puts the device into a sleep mode.

In FIG. 5, the control panels 500A and 500B of devices 101 and 102, respectively are communicatively coupled via cable 508. Such a cable may be used for any of a variety of reasons such as, for instance, to share power between the two devices. Signals from the detectors can be communicated to a remote device for processing using a wired or wireless communication protocol. Antenna 509 enables each of the devices to communicate to each other and/or to a remote device such as, for example, a smartphone, a Wi-Fi router, an I-Pad, a Tablet-PC, a laptop, a computer, and the like, over network 510, shown as an amorphous cloud for illustrative purposes. Such communication may utilize a Bluetooth protocol. The present devices are also capable of communicating text, email, picture, graph, chart, and/or a pre-recorded message over network 510. A microprocessor (CPU) or ASIC, internal to the control panels, may be configured to activate the illuminators to emit light for a desired length of time; turn the illuminators ON/OFF according to a pre-defined interval; turn the devices ON/OFF according to a pre-defined schedule; change a wavelength band of any of the illuminators; adjust a sensitivity of any of the sensors; and receive signal for processing such that the PTT for the subject can be determined.

It should be appreciated that the embodiments described are illustrative and are not to be viewed as limiting the scope of the appended claims solely to the configuration shown.

### Example Flow Diagram

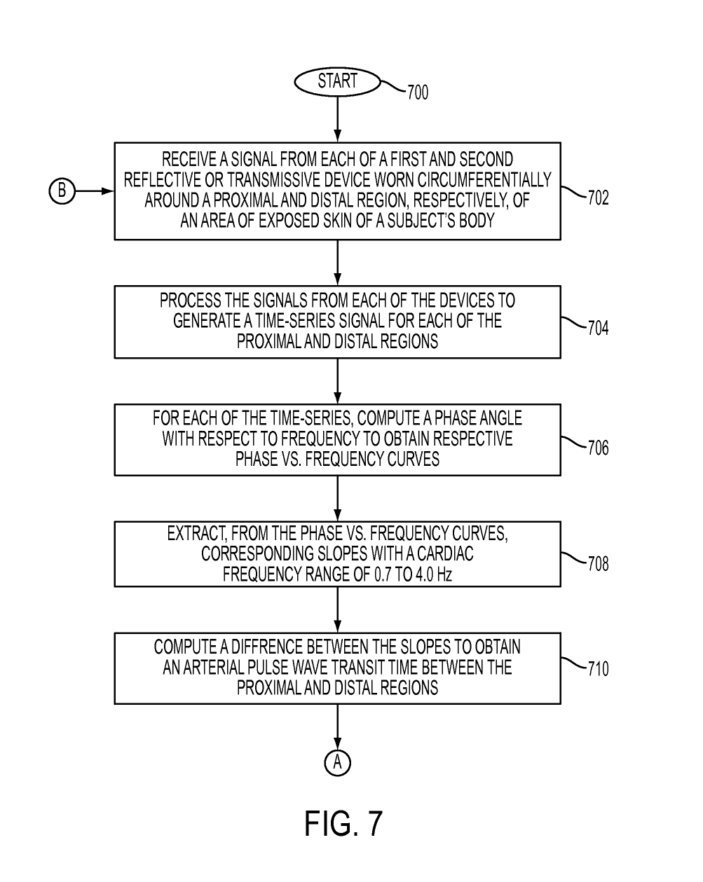

Reference is now being made to the flow diagram of FIG. 7 which illustrates one example embodiment of the present method for determining arterial pulse wave transit time in accordance with the teachings disclosed herein. Flow processing begins at 700 and immediately proceeds to step 702.

At step 702, receiving a signal from each of a first and second reflective or transmissive device worn circumferentially around a proximal and a distal region, respectively, of an area of exposed skin of a subject's body. The signals are received from sensors of each of the “proximal” and “distal” devices worn by the subject. The devices may be any of the transmissive or reflective devices of FIGS. 2 and 3.

At step 704, process the signals from each of the devices to generate a time-series signal for each of the proximal and distal regions.

At step 706, compute, for each of the time-series signals, a phase angle with respect to frequency to obtain respective phase v/s frequency curves.

At step 708, extract, from the phase v/s frequency curves, slopes within a cardiac frequency range of 0.75 to 4.0 Hz.

At step 710, compute a difference between the extracted slopes to obtain an arterial pulse wave transit time between the proximal and distal regions. The arterial pulse wave transit time, including any signals, interim values, formulae, and the like, may be stored to a storage device or communicated to a remote device over a network.

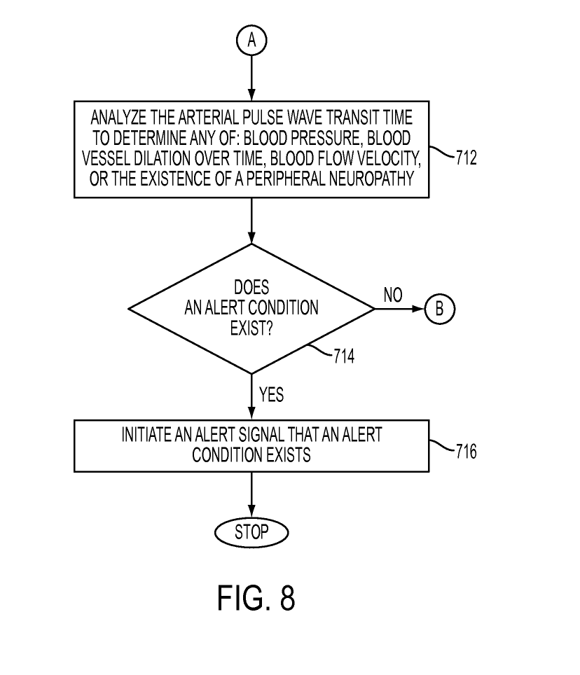

Reference is now being made to FIG. 8 which is a continuation of the flow diagram of FIG. 7 with flow processing continuing with respect to node A.

At step 712, analyze the arterial pulse wave transit time to determine any of: blood pressure, blood vessel dilation over time, blood flow velocity, or the existence of a peripheral vascular disease.

At step 714, a determination is made whether an alert condition exists. Such a determination can be made using, for example, one or more threshold levels which may be retrieved from a memory, storage device, or data base, or which may be set or pre-set by a technician or medical professional. The alert condition can be determined by a visual examination of the received measurement signals, the arterial pulse wave transit time, or by an algorithm monitoring these signals or the results obtained from processing those signals. If it is determined that an alert condition exists then processing continues with respect to step 716.

At step 716, an alert signal is initiated. The alert signal can be sent to a technician, nurse, medical practitioner, and the like. In one embodiment, the alert signal is communicated by the device's control panel via network 510 using communication element 509. Such a signal may take the form of a tone or ring, or a visual or audible message being activated at a nurse's station that the patient requires attention. The alert may take the form of a message such as a text, audio, and video message. The alert may take the form of a blinking colored light on the device's control panel. In this embodiment, further processing stops. Otherwise, processing continues with respect to node B wherein, at step 702, signals are continuously received and processed in a similar manner.

It should also be appreciated that the flow diagrams depicted herein are illustrative. One or more of the operations may be performed in a differing order. Other operations may be added, modified, enhanced, or consolidated. Variations thereof are intended to fall within the scope of the appended claims.

### Example Networked System

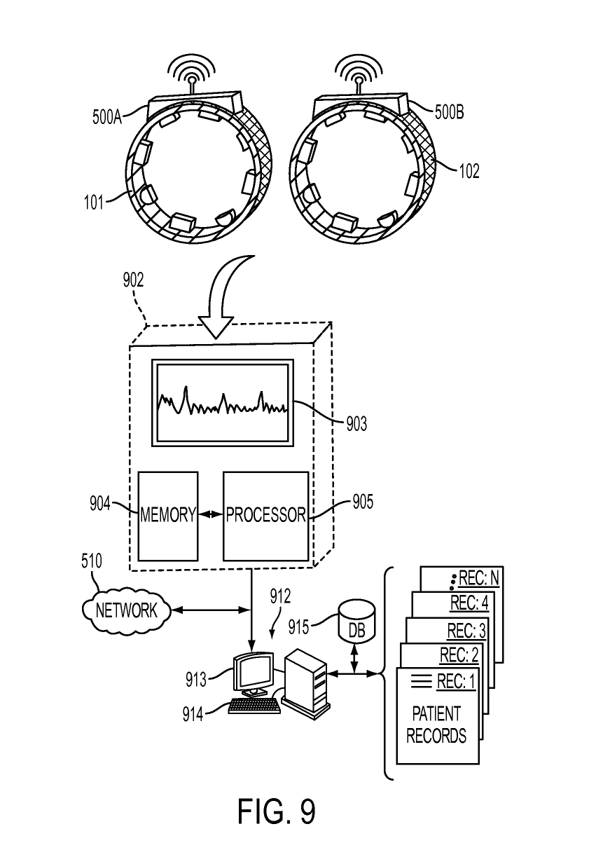

Reference is now being made to FIG. 9 which illustrates a block diagram of one example signal processing system 900 for performing various aspects of the teachings hereof as discussed with respect to the flow diagrams of FIGS. 7 and 8.

The control panels 500A and 500B, of each of the devices proximally and distally on the subject′ body, utilize their respective communication elements to communicate their respective signals to a wireless smartphone device 902. Smartphone 902 has a display 903, a memory 904, and a processor 905 which executes machine readable program instructions for analyzing the received signals to determine an arterial pulse wave transit time for the subject. The smartphone is configured to work with various embodiments of the present transmissive or reflective sensing devices. Some or all of the processing and functionality performed by the smartphone may be performed entirely with any of the control panels 500A-B or with the workstation 912.

The smartphone is in communication with a workstation 912 comprising a computer case housing a motherboard, CPU, memory, interface, storage device, and a communications link such as a network card. The workstation has a display device 913 such as a CRT, LCD, or touchscreen display. An alphanumeric keyboard 914 and a mouse (not shown) effectuate a user input. The workstation has an operating system and other specialized software configured to display a variety of numeric values, text, scroll bars, pull-down menus with user selectable options, and the like, for entering, selecting, or modifying information. The workstation has a removable media (not shown).

The workstation implements a database 915 wherein various patient records are stored. Information obtained using the present devices can be stored to patient records. Records stored in the database can be indexed, searched, and retrieved in response to a query. Patient information can be stored to any of the records in the database and used for physiological event monitoring. Although the database is shown as an external device, the database may be internal to the workstation mounted on a hard disk housed in the computer case. Processor 905 and memory 904 are in communication with the workstation via pathways (not shown) and may further be in communication with one or more remote devices over network 510. Some or all of the functionality performed by the smartphone device 902 may be performed, in whole or in part, by the workstation.

Various aspects of the teachings hereof may be practiced in distributed environments where tasks are performed by a plurality of devices linked via a network and may be implemented using any known or later developed systems, structures, devices, or software by those skilled in the applicable arts without undue experimentation from the description provided herein.

One or more aspects of the systems and methods described herein are intended to be incorporated in an article of manufacture which may be shipped, sold, leased, or otherwise provided separately either alone or as part of a product suite. The above-disclosed features and functions or alternatives thereof, may be combined into other systems or applications. Presently unforeseen or unanticipated alternatives, modifications, variations, or improvements may become apparent and/or subsequently made by those skilled in the art and, further, may be desirably combined into other different systems or applications. Changes to the above-described embodiments may be made without departing from the spirit and scope of the invention.

The teachings of any printed publications including patents and patent applications, are each separately hereby incorporated by reference in their entirety.

What is claimed is:

1. A method for determining arterial pulse wave transit time, the method comprising:
receiving a signal from each of a first and second device worn on a proximal and a distal region, respectively, of a subject's body, each device comprising at least one emitter/detector pair fixed to an inner side with at least one detector being paired to at least one illuminator, each detector comprising at least one sensor that is sensitive to a wavelength band of light emitted by one of said illuminators, each device generates signals that are proportional to an intensity of light emitted by its illuminator, each emitter/detector pair being separated from its respective paired at least one illuminator by distance D; and
analyzing said signals to determine an arterial pulse wave transit time for said subject between said proximal and distal regions.

2. The method of claim 1, wherein at least one of said devices is a transmissive device, said distance D defining a chord of living tissue through which light emitted by an illuminator passes, said distance being less than 75% of a diametrical distance of an area where said device is worn, each sensor measuring an intensity of light passing through a chord of living tissue.

3. The method of claim 1, wherein at least one of said devices is a reflective device, said distance D defining a distance between each illuminator and paired detector as measured around said circumference, light emitted by an illuminator impacting a surface of skin at an angle θ_{L}, each sensor measuring an intensity of light reflecting off said skin surface at an angle θ_{R}, where 0°<(θ_{L}, θ_{R})<90°.

4. The method of claim 1, wherein a wavelength band of said illuminators is centered around any of: 660 nm and 940 nm.

5. The method of claim 1, wherein analyzing said signals to determine an arterial pulse wave transit time comprises:
processing signals from each of said devices to generate a time-series signal for each of said proximal and distal regions;
computing, for each of said time-series signals, a phase angle with respect to frequency to obtain a phase v/s frequency curves after phase unwrapping;
extracting, from said phase v/s frequency curves, slopes within a cardiac frequency range of 0.75 to 4.0 Hz; and
computing a difference between said extracted slopes, said difference comprising said arterial pulse wave transit time between said proximal and distal regions.

6. The method of claim 1, wherein analyzing said signals to determine an arterial pulse wave transit time comprises:
analyzing signals from each of said devices to generate a phase difference; and
processing said phase difference with said subject's heart rate to determine said arterial pulse wave transit time between said proximal and distal regions.

7. The method of claim 1, further comprising increasing an accuracy of measurements by performing any of:
band pass filtering signals to restrict frequencies of interest;
detrending signals to remove non-stationary components;
averaging signals to obtain a composite signal, discarding signals;
weighting signals based on a statistical analysis; and
discarding intensity values determined to be below a level of acceptability.

8. The method of claim 1, further comprising any of:
activating said illuminators to emit light for a desired length of time;
turning said illuminators ON/OFF according to a pre-defined interval;
turning said bands ON/OFF according to a pre-defined schedule;
changing a wavelength band of any of said illuminators; and
changing a sensitivity of any of said sensors.

9. The method of claim 1, further comprising analyzing said arterial pulse wave transit time to determine any of:
a blood pressure in said subject's vascular network;
an amount of blood vessel dilation over time;
a blockage of blood flow;
blood flow velocity; and
an existence of a peripheral vascular disease

10. The method of claim 1, further comprising communicating said arterial pulse wave transit time to a remote device using any of: a wired and a wireless connection.

11. The method of claim 10, wherein said communication comprises any of: text, email, picture, graph, chart, and pre-recorded message.

12. A system for determining arterial pulse wave transit time, the system comprising:
a first and second device worn on a proximal and a distal region, respectively, of a subject's body, each device comprising at least one emitter/detector pair fixed to an inner side with at least one detector being paired to at least one illuminator, each detector comprising at least one sensor that is sensitive to a wavelength band of light emitted by one of said illuminators, each device communicating signals that are proportional to an intensity of light emitted by its illuminator, each emitter/detector pair being separated from its respective paired at least one illuminator by distance D; and
a processor in communication with said devices, said processor executing machine readable program instructions for analyzing said sensor signals to determine an arterial pulse wave transit time for said subject between said proximal and distal regions.

13. The system of claim 12, wherein at least one of said devices is a transmissive device, said distance D defining a chord of living tissue through which light emitted by an illuminator passes, said distance being less than 75% of a diametrical distance of an area where said device is worn, each sensor measuring an intensity of light passing through a chord of living tissue.

14. The system of claim 12, wherein at least one of said devices is a reflective device, said distance D defining a distance between each illuminator and paired detector as measured around said circumference, light emitted by an illuminator impacting a surface of skin at an angle θ_{L}, each sensor measuring an intensity of light reflecting off said skin surface at an angle θ_{R}, where 0°<(θ_{L}, θ_{R})<90°.

15. The system of claim 12, wherein a wavelength band of said illuminators is centered around any of: 660 nm and 940 nm.

16. The system of claim 12, wherein analyzing said signals to determine an arterial pulse wave transit time comprises:
processing signals from each of said devices to generate a time-series signal for each of said proximal and distal regions;
computing, for each of said time-series signals, a phase angle with respect to frequency to obtain a phase v/s frequency curves;
extracting, from said phase v/s frequency curves, slopes within a cardiac frequency range of 0.75 to 4.0 Hz; and
computing a difference between said extracted slopes, said difference comprising said arterial pulse wave transit time between said proximal and distal regions.

17. The system of claim 12, wherein analyzing said signals to determine an arterial pulse wave transit time comprises:
analyzing signals from each of said devices to generate a phase difference; and
processing said phase difference with said subject's heart rate to determine said arterial pulse wave transit time between said proximal and distal regions.

18. The system of claim 12, said processor further increasing an accuracy of measurements by performing any of:
band pass filtering signals to restrict frequencies of interest;
detrending signals to remove non-stationary components;
averaging signals to obtain a composite signal, discarding signals;
weighting signals based on a statistical analysis; and
discarding intensity values determined to be below a level of acceptability.

19. The system of claim 12, said processor further performing any of:
activating said illuminators to emit light for a desired length of time;
turning said illuminators ON/OFF according to a pre-defined interval;
turning said bands ON/OFF according to a pre-defined schedule;
changing a wavelength band of any of said illuminators; and
changing a sensitivity of any of said sensors.

20. The system of claim 12, further comprising analyzing said arterial pulse wave transit time to determine any of:
a blood pressure in said subject's vascular network;
an amount of blood vessel dilation over time;
a blockage of blood flow;
blood flow velocity; and
an existence of a peripheral vascular disease.

21. The system of claim 12, wherein at least one of said devices further comprises a connection for receiving power from a power source.

22. The system of claim 12, said processor communicating said arterial pulse wave transit time to a remote device using any of: a wired and a wireless connection.

23. The system of claim 22, wherein, in response to said determination, said remote device communicating any of: text, email, picture, graph, chart, and pre-recorded message.

24. The system of claim 22, wherein said remote device is any of: a smartphone, an iPad, a tablet-PC, a laptop, a router, a server, and a computer workstation.

25. The system of claim 24, wherein said remote device further comprises any of: a USB connection, a micro-HDMI connection, a transmitter, a receiver, a display, a memory, a storage device, and a connection for delivering power to said apparatus.
